(19) United States
(12) Patent Application Publication
(10) Pub. No.: US 2018/0333508 A1
(43) Pub. Date: Nov. 22, 2018

(54) IMAGING PROBE FOR ANGIOGENIC ACTIVITY IN PULMONARY ARTERIAL HYPERTENSION

(71) Applicant: THE BRIGHAM AND WOMEN'S HOSPITAL, INC., Boston, MA (US)

(72) Inventors: Paul B Yu, Boston, MA (US); Marcelo Dicarli, Needham, MA (US)

(21) Appl. No.: 15/770,994

(22) PCT Filed: Nov. 3, 2016
(86) PCT No.: PCT/US16/60383

§ 371 Date: Apr. 25, 2018

Related U.S. Application Data
(60) Provisional application No. 62/250,102, filed on Nov. 3, 2015.

(51) Int. Cl. A61K 51/10; A61B 5/055; A61B 6/03; G01R 33/56; G01T 1/29

(52) U.S. Cl. CPC A61K 51/103; A61B 5/055; A61B 6/032; A61B 5/4884; G01R 33/5601; G01T 1/2985; A61B 6/037

(57) ABSTRACT

A method of detecting a disease associated with pulmonary vascular remodeling. The method includes administering a radioisotope-conjugated antibody against vascular endothelial growth factor (VEGF). The method further includes imaging said antibody using positron emission tomography (PET), computed tomography (CT), or magnetic resonance imaging (MIR). Retention of said antibody reflects vascular remodeling.

### CROSS-REFERENCE(S) TO RELATED APPLICATION(S)

This application claims priority to U.S. Patent Application No. 62/250,102, filed Nov. 3, 2015, which is hereby incorporated by reference in its entirety.

### FIELD OF THE INVENTION

The present invention relates generally to an imaging probe for angiogenic activity in pulmonary arterial hypertension. More particularly, the present invention relates to a pulmonary vascular biomarker comprising a radioisotope-conjugated antibody (e.g., ⁸⁹[Zr]-bevacizamub), wherein the biomarker is used for detecting a disease associated with pulmonary vascular remodeling or for monitoring the efficacy of therapeutics or prophylactics for a disease that is associated with pulmonary vascular remodeling.

### BACKGROUND

Pulmonary hypertension (PH) describes a heterogeneous spectrum of diseases characterized by increased pulmonary vascular resistance. Reflecting the diverse causes and phenotypes of PH, the World Health Organization (WHO) system classifies PH disease in five groups, including Group I pulmonary arterial hypertension (PAH), as well as PH associated with left heart disease, PH associated with chronic lung disease, chronic thromboembolic PH, and other miscellaneous types of PH.

WHO Group I PAH is a disorder of elevated pulmonary vascular resistance characterized by progressive remodeling and obliteration of resistance-determining vessels of the pulmonary circulation. It is defined as a sustained elevation in mean pulmonary arterial pressure of at least about 25 mmHg, pulmonary vascular resistance greater than about 240 dyne·s/cm⁵, and pulmonary capillary wedge pressure less than about 15 mmHg in the absence of significant left ventricular or valve dysfunction, lung disease, or thrombotic disease. Group I PAH may be associated with connective tissue disease, amphetamine use, HIV, or congenital heart disease and may be familial or idiopathic.

A study of more than 10,000 U.S. Veterans revealed a surprisingly high prevalence of all types of PH (3.2%) based on screening echocardiograms and a relatively low rate of recognition (17.3%) by providers. Nearly half of patients were deceased at an average of less than 3 years after the index echo, which was consistent with National Institutes of Health (NIH) registry data from the 1980s showing a median survival of 2.8 years following diagnosis, highlighting the continued need for improved diagnosis and treatment in the current era. Outcomes could likely be improved with earlier diagnosis and deployment of therapies that directly target pulmonary vascular remodeling before irreversible changes have occurred.

PH can present insidiously, with initial symptoms that include dyspnea and fatigue, followed by more advanced symptoms such as edema, chest pain, and/or syncope. Given the overlap of these symptoms with other cardiac and pulmonary conditions, the diagnosis of PAH is often delayed until clinical suspicion is raised and diagnosis is confirmed by right heart catheterization. Despite increased awareness among clinicians, delays generally ranging from about 2 to about 4 years from the onset of symptoms and a mean of about 2 alternative diagnoses entertained prior to the diagnosis of PAH are commonly reported. This delay in diagnosis may be associated with a worsened prognosis, possibly due to the unmitigated progression of disease towards an irreversible and treatment-unresponsive state. It has been proposed that significant pruning or obliteration of the pulmonary circulation may be present by the time PH is confirmed by catheterization, owing to the fact that the large physiologic reserve of this highly parallel circuit can mask early disease. In other words, poor survival in this disease might reflect that PAH is a relatively late manifestation of the underlying pulmonary vascular disease process.

Untreated, PH carries high mortality, generally from progression to right heart failure. Current therapies include several classes of vasodilators, including prostacyclin, calcium channel blockers, PDE5 inhibitors, endothelin receptor antagonists (ETRA), and/or the generally soluble guanylate cyclase stimulator riociguat. While some of these medications may be delivered to airways by inhalation to enhance local pulmonary effects, none of these medications is inherently selective for the pulmonary vasculature, and, thus, systemic vasodilatation, hypotension, and toxicities in other organs limit their use and must be monitored. Patients may be unresponsive to certain agents at presentation or during the course of treatment due, e.g., to non-vasoreactive disease, disease progression, and/or drug tachyphylaxis.

Tailoring PAH therapy represents an ongoing challenge with few objective biomarkers for guidance besides functional status, NT-proBNP measurements, and invasive hemodynamics—all of which reflect late manifestations of disease progression. Currently available treatments for PH have yielded improvements in function and modest improvements in mortality but act principally as vasodilators rather than to inhibit remodeling. With current PH therapies, survival is approximately 57% at 5 years following diagnosis, with high mortality due to progression to right heart failure.

Delayed diagnosis, the lack of more direct biomarkers of disease activity, and/or the lack of treatments that can arrest or reverse pulmonary vascular remodeling are all barriers to improved outcomes in PH-diagnoses that might be addressed by a novel imaging modality detecting angiogenic activity in PH. It would be desirable to have a sensitive imaging test that could detect early pathologic changes in the pulmonary vasculature to expedite diagnosis over current algorithms, which lead to PH only by a process of elimination, and could potentially detect pulmonary vascular disease prior to the development of hemodynamically significant PAH. A sensitive and specific imaging test could stratify disease severity and risk, tailor pharmacotherapy, and validate experimental therapies acting by entirely novel mechanisms to arrest or reverse disease progression.

The use of ⁸⁹Zr-bevacizumab as an imaging probe for PAH disease activity would represent a considerable breakthrough as well as a challenge to the current diagnostic and therapeutic paradigm. Contemporary management of PAH has relied upon surrogate biomarkers such as exercise function, invasive hemodynamic measurements, and circulating biomarkers such as NT-proBNP, all of which are, at best, indirect measurements of disease activity and treatment response, necessitated by the fact that, under normal circumstances, pulmonary vascular tissues are inaccessible for tissue diagnosis or for informing disease status. Due to the inaccessibility of the vasculature, invasive testing via catheterization, or biochemical or imaging evidence of right ventricular strain, hypertrophy or failure are typical means of testing and generally only reflect advanced disease with end-organ damage.

There are currently no widely accepted imaging biomarkers of pulmonary vascular disease. As such, use of ⁸⁹Zr-bevacizumab PET as a pulmonary vascular imaging modality could have considerable short-term and long-term impacts on PAH care. Short-term applications of this type of probe include: (1) assessing treatment responses rapidly when tailoring a regimen of approved therapies; (2) screening for early pathogenetic changes in individuals at high risk for PAH, i.e., individuals with severe liver dysfunction, individuals with significant exposure to PAH-causing toxins (e.g., anorexigens, methamphetamine, or the like), individuals with scleroderma, CREST syndrome, systemic lupus erythematosus, rheumatoid arthritis, mixed connective tissue disease, other conditions at elevated risk for PAH, and/or individuals with a family history of PAH; and/or (3) expedited screening for pulmonary vascular disease in individuals with dyspnea and with normal pulmonary function tests, cardiac stress testing, resting echocardiograms, and resting invasive hemodynamic measurements that are non-diagnostic for PAH. The possibility of discordant findings between ⁸⁹Zr-bevacizumab PET and invasive hemodynamic measurements suggests a novel clinical entity of pre-hypertensive, early pulmonary vascular disease—an entity which might have a distinct natural history and greater response to therapy than established PAH and which could provide a rationale for early intervention. Long-term impacts of this novel diagnostic modality could include, for example: (1) the identification of novel, disease-modifying therapies based on their capacity to normalize pulmonary vascular endothelial growth factor (VEGF) expression; and/or (2) the definition of novel sub-phenotypes or clinical stages based on the presence or absence of VEGF. Excessive VEGF expression in the pulmonary vasculature appears to be a consistent feature of human and experimental PAH, which may reflect a process of disordered angiogenesis that is coupled to disease progression. It would be desirable and clinically useful to confirm coupling of this angiogenic marker to disease activity and treatment responses using this imaging biomarker.

Thus, there exists a need for deploying current agents earlier in the disease course of PH or by introducing novel agents which directly target vascular remodeling.

### SUMMARY OF THE INVENTION

According to one embodiment, a method of detecting a disease associated with pulmonary vascular remodeling comprising administering a radioisotope-conjugated antibody against vascular endothelial growth factor (VEGF). The method further comprises imaging said antibody using positron emission tomography (PET), computed tomography (CT), or magnetic resonance imaging (MIR). Retention of said antibody reflects vascular remodeling.

According to another embodiment, a method of monitoring the efficacy of therapeutics or prophylactics for a disease that is associated with pulmonary vascular remodeling comprises administering a radioisotope-conjugated antibody against vascular endothelial growth factor (VEGF). The method further comprises imaging said antibody using positron emission tomography (PET), computed tomography (CT), or magnetic resonance imaging (MIR). Said imaging reflects the ability of said therapeutics or prophylactics to decrease said vascular remodeling.

According to another embodiment, a method of identifying novel therapeutics for a disease that is associated with pulmonary vascular remodeling comprises administering a radioisotope-conjugated antibody against vascular endothelial growth factor (VEGF) and imaging said antibody using positron emission tomography (PET), computed tomography (CT), or magnetic resonance imaging (MIR). Said imaging reflects the ability of said novel therapeutics to decrease said vascular remodeling.

According to another embodiment, a pulmonary vascular biomarker comprises a radioisotope-conjugated antibody. The biomarker is used for detecting a disease associated with pulmonary vascular remodeling or for monitoring the efficacy of therapeutics or prophylactics for a disease that is associated with pulmonary vascular remodeling.

Additional aspects of the invention will be apparent to those of ordinary skill in the art in view of the detailed description of various embodiments, a brief description of which is provided below.

### BRIEF DESCRIPTION OF THE DRAWINGS

Various advantages of the invention will become apparent upon reading the following detailed description and upon reference to the drawings.

FIG. 1 shows images of lungs explanted from a patient with heritable PAH (top left panel), remodeled small vessels in monocrotaline (MCT) treated rats (top right panel), lung tissue from an idiopathic PAH patient (middle left), medial and adventitial areas of remodeled vessels of an SU5416-hypoxia (SU-Hx) treated rat (middle right panel), a human control treated with bevacizumab (bottom left), and a rat control treated with bevicizimab (bottom right).

FIG. 2 shows the sensitivity of bevacizumab recognition of immunoblotted human, rat and mouse VEGF-A.

FIG. 3 includes images showing immunfluorescence of serial 10 μm lung sections from rats treated with SU-Hx or control rats stained with either a pan-species specific anti-VEGF mAb (top panels) or bevacizumab (middle panels).

FIG. 4 shows representative PET-CT scans of rats subjected to SU5416 and hypoxia (SU-Hx, right panels) versus control rats (left panels).

FIG. 5A show representative regions of interest (ROIs) used to define peripheral lung fields and mediastinal structures using a computed tomography (CT) image.

FIG. 5B show representative ROIs used to define peripheral lung fields and mediastinal structures using PET data super-imposed with the CT image of FIG. 5A.

FIG. 6 shows autoradiography images of rat lung sections demonstrating enhanced retention of ⁸⁹Zr-bevacizumab among rats with SU-Hx induced PAH or diseased rats receiving dual vasodilator treatment as compared to control rats.

FIG. 7 shows histology comparing neointimal and complex lesions of medium vessels in SU-Hx and control rat lungs.

FIG. 8A shows the effect of variable single doses of MCT administered to adult Sprague-Dawley rats resulting in varying degrees of PAH at 3 weeks based on right ventricular systolic pressure measurements.

FIG. 8B shows the effect of variable single doses of MCT administered to adult Sprague-Dawley rats resulting in variable degrees of right ventricular hypertrophy, expressed as Fulton's ratio.

FIG. 9A is a plot showing the development of elevated right ventricular systolic pressure (RVSP) as a function of time following a single 40 mg/kg s.c. dose of MCT.

FIG. 9B is a plot of right ventricular hypertrophy (RV/(LV+S)) as a function of time following a single 40 mg/kg s.c. dose of monocrotaline.

FIG. 10A is a plot showing adult rats treated with MCT that were administered TGFBRII-Fc or vehicle for 3 weeks where TGFBRII-Fc significantly attenuated RVSP.

FIG. 10B is a plot showing adult rats treated with MCT that were administered TGFBRII-Fc or vehicle for 3 weeks with RV hypertrophy in comparison to vehicle.

FIG. 10C is a plot showing adult rats treated with MCT that were administered TGFBRII-Fc or vehicle for 3 weeks with decreased percentage of fully muscularized vessels.

FIG. 10D is a plot showing adult rats treated with MCT that were administered TGFBRII-Fc or vehicle for 3 weeks with decreased medial wall thickness.

FIG. 10E is a plot showing that TGFBRII-Fc treatment generally reduced muscularization, evidenced by smooth muscle actin staining of vWF⁺ small vessels.

FIG. 10F shows a series of images indicating that delayed treatment with TGFBRII-Fc starting at 2.5 weeks after MCT generally improved survival.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

The embodiments described herein generally relate to an imaging probe for angiogenic activity in pulmonary arterial hypertension. More particularly, the present invention relates to a pulmonary vascular biomarker comprising a radioisotope-conjugated antibody (e.g., ⁸⁹[Zr]-bevacizamub), wherein the biomarker is used for detecting a disease associated with pulmonary vascular remodeling or for monitoring the efficacy of therapeutics or prophylactics for a disease that is associated with pulmonary vascular remodeling.

The embodiments described herein relate generally to a comprehensive pre-clinical development program for ⁸⁹Zr-bevacizumab, a radioisotope-conjugated humanized monoclonal antibody directed against vascular endothelial growth factor (VEGF) as a positron emission tomography (PET) imaging probe for the diagnosis and management of PAH. The embodiments discussed herein implement the theory that PAH may arise from dysregulated angiogenic activity in the pulmonary vasculature. Consistent with VEGF's tendency to be overexpressed in the lesions of human and experimental PAH, the embodiments described herein show that ⁸⁹Zr-bevacizumab is retained avidly in remodeled small pulmonary arterioles and vascular lesions of experimental PAH, but not control animals, and binds avidly to vascular lesions in sections from human lungs with PAH, but not healthy controls. As such, ⁸⁹Zr-bevacizumab in distal lung vessels appears to be a sensitive marker of pulmonary vascular remodeling that may directly reflect PAH disease activity. This PET molecular imaging probe may have the capacity to detect disease in its earliest stages, reveal the progression of disease and disease burden, and/or predict positive treatment responses. Gaining direct insight into disease activity could help to identify novel treatments that alter remodeling, rather than acting strictly as vasodilators.

The embodiments described herein may be used to address several principles for improving outcomes in PAH including earlier diagnosis, sensitive and non-invasive testing of disease activity, and/or identifying interventions that can arrest or reverse pulmonary vascular remodeling. This modality would represent the first molecular imaging agent validated for the diagnosis and management of PAH. The experiments described herein were designed to assess the sensitivity and specificity of this agent in early and advanced experimental PAH, its ability to detect lesions in human PAH lungs ex vivo, and its ability to discriminate between the impact of current vasodilator therapies versus novel experimental therapies that directly modify vascular remodeling based on the modulation of bone morphogenetic protein (BMP) and transforming growth factor-β (TGF-β) signaling.

Approach

PAH exhibits features of disordered angiogenesis and abnormal angiogenic signaling. The histopathology of PAH includes hypertrophy of the medial smooth muscle, concentric and obstructive lesions of small (less than about 50 μm) resistance-determining arterioles, and complex, multi-channeled plexiform lesions within arterioles. Obstructive and concentric lesions exhibit a neointima consisting of actively proliferating myofibroblast and endothelial lineages, while plexiform lesions exhibit a generally heterogeneous structure with proliferating endothelial cells (EC) in the periphery and quiescent EC in the core and lining luminal structures, interspersed with smooth muscle α-actin positive lineages. The architecture, composition, and dynamic nature of PAH vascular lesions has prompted their comparison to a process of “disordered angiogenesis.”

Evidence of dysregulated angiogenic signaling lends additional support for the disordered angiogenesis hypothesis in PAH. Studies demonstrate that vascular endothelial growth factor and its receptor VEGFR2 are overexpressed at mRNA and protein levels in obstructive and plexiform lesions of human PAH_ENREF_15 and in experimental PAH. In fact, circulating VEGF and other potent angiogenic modulatory molecules, Angiopoietins-1 and -2 and basic fibroblast growth factor (bFGF) are observed to be elevated in individuals with diverse etiologies of Group I PAH. It was found that the soluble isoform of VEGFR1, which can act as an endogenous VEGF ligand trap, is increased in the circulation of individuals with diverse etiologies of Group I PAH, correlating inversely with functional status and transplant-free survival. In contrast to NT-proBNP, VEGFR1 was elevated even in patients with minimally symptomatic PAH (NYHA class I-II). The consistency of these observations supports the notion that dysregulated angiogenic signaling is a hallmark of PAH and perhaps is even a marker of early disease. Initially reported in Group I PAH, these findings may likewise apply in other forms of PH, given the shared histopathologic features of these diverse diseases.

Validation of VEGF as a Molecular Imaging Target in Experimental and Human PAH.

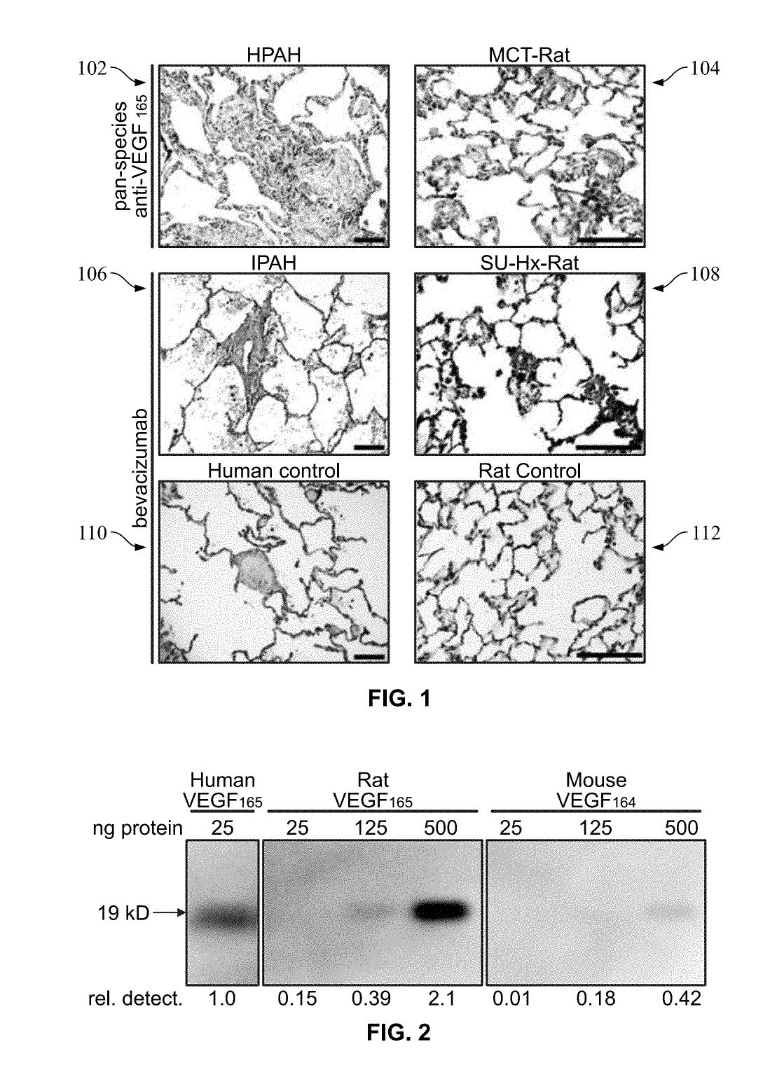

By serial PET imaging and invasive hemodynamic measurements, the coupling of ⁸⁹Zr-bevacizumab lung imaging to disease progression was tested in two longitudinal models of experimental PAH. The binding of systemically administered ⁸⁹Zr-bevacizumab to diseased but not healthy lung vascular tissues was demonstrated in vivo by PET imaging and ex vivo by autoradiography and immunofluorescence. Specificity of ⁸⁹Zr-bevacizumab or bevacizumab for the vascular lesions of human PAH was determined by staining human lung tissues from PAH patients and from healthy control lungs. VEGF is overexpressed in the lesions of human and experimental PAH. The VEGF family has several members, of which VEGF-A plays the most significant role in angiogenesis, potently inducing proliferation, migration, and survival of EC, and triggering lumen formation and vascular permeability. VEGF-A includes several isoforms resulting from alternative splicing, the most widely expressed and potent isoform being VEGF₁₆₅. It was found that two well-validated monoclonal antibodies (mAb) detect strong VEGF₁₆₅ immunoreactivity in neointimal and plexiform lesions of human Group I PAH, but minimal and sporadic expression in normal lung vasculature, as shown in FIG. 1. One of these antibodies, bevacizumab, is a humanized mAb designed to block human VEGF-A, an approved therapy for colorectal carcinoma, renal cell carcinoma, non-small cell lung cancer, and platinum-resistant ovarian cancer, with an extensive body of human safety and tolerability data.

Referring to FIG. 1, images 102, 104 show that explanted lung from a patient with heritable PAH (HPAH) exhibits strong VEGF immunoreactivity in the luminal areas of plexiform lesions, and diffuses reactivity in alveoli and large airway epithelium (image 102) using a pan-species monoclonal anti-VEGF₁₆₅ antibody. Remodeled small vessels in monocrotaline (MCT)-treated rats similarly exhibit luminal and medial immunoreactivity, as shown in image 104. Images 106, 108 show, using bevacizumab, lung tissue from an idiopathic PAH patient exhibits strong VEGF immunoreactivity in luminal, medial, and adventitial areas of remodeled vessels (image 106), as do remodeled small vessels of SU5416-hypoxia (SU-Hx) treated rats (image 108). Images 110, 112 show that staining of small and medium sized vessels exhibit weaker and patchy reactivity with bevacizumab in normal human (image 110) and normal rat (image 112) lung tissues (bars=100 μm).

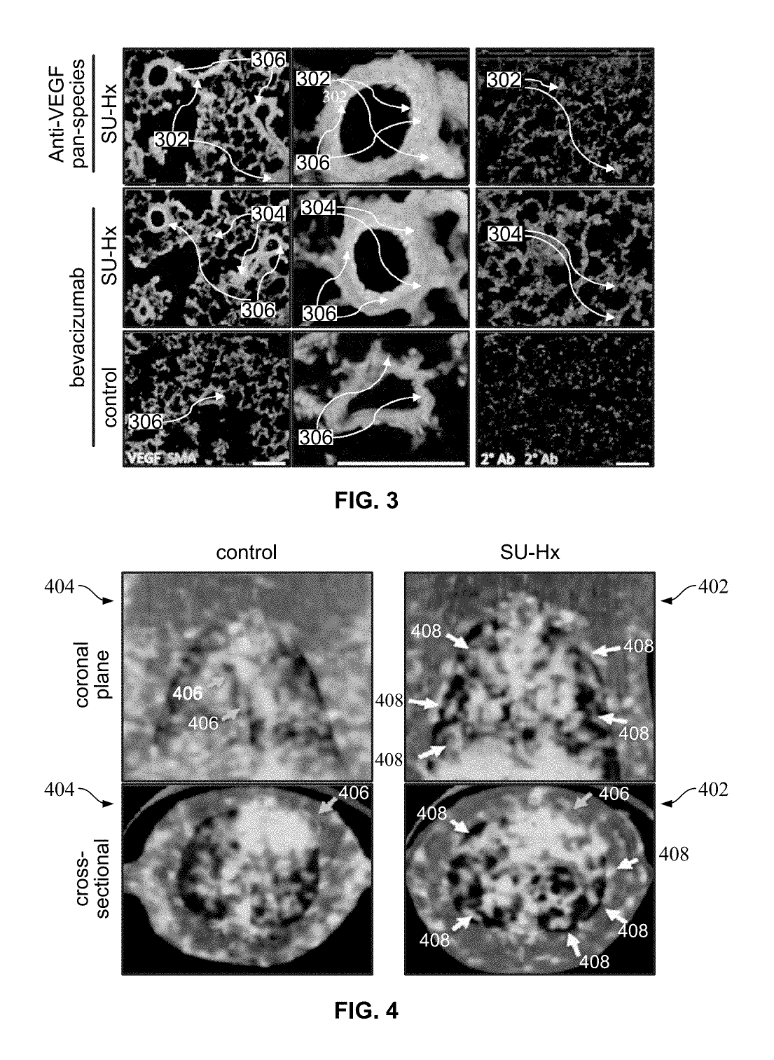

In addition to recognizing human VEGF₁₆₅, bevacizumab also recognizes rat VEGF₁₆₅, albeit with 8-10 fold decreased sensitivity, and recognizes murine VEGF₁₆₄ with about 50-fold less sensitivity (see FIG. 2). Referring back to FIG. 1, to test whether or not VEGF overexpression is also a hallmark of experimental PAH, two rat models were utilized—the SU-Hx rat PAH model, and the monocrotaline (MCT) induced model. Adult Sprague-Dawley rats treated with a single dose of MCT (about 40 mg/kg s.c.) developed significant PAH and distal arteriolar muscularization after about 3 weeks and exhibited enhanced VEGF expression in remodeled small arterioles (see image 104 of FIG. 1). Adult Sprague-Dawley rats treated with SU5416 (about 20 mg/kg/week s.c) in combination with normobaric hypoxia (FIO₂=0.10) for about 3 weeks followed by normoxia for about 2 weeks developed several histopathologic features of severe human PAH, including severe medial thickening, neointimal formation, obstructive lesions, and complex, plexiform-like lesions (see image 108 of FIG. 1). Importantly, both a pan-species specific anti-VEGF-A mAb and bevacizumab showed enhanced staining of small (less than about 50 μm) vessels and neointima in SU-Hx rats, but not in the controls (see FIG. 3).

FIG. 3 shows the immunfluorescence of serial 10 μm lung sections from rats treated with SU-Hx or control rats stained with either a pan-species specific anti-VEGF mAb 302 or bevacizumab 304, and smooth muscle a-actin specific mAb 306 reveal medial thickening of medium sized vessels and enhanced intimal, medial and adventitial staining for VEGF in SU-Hx rats. Slides stained with fluorescent secondary antibodies only are shown on the right (bars=100 μm).

The SU-Hx rat model of PAH is considered unique in its ability to recapitulate in a small animal model histological findings of angio-proliferative and angio-obliterative remodeling reminiscent of human PAH. SU-Hx rats overexpress VEGF-A in their pulmonary vascular lesions like human PAH, which is consistent with the angio-proliferative phenotype of this model. MCT-treated rats also exhibit enhanced VEGF-A expression, but in a generally more limited anatomic distribution than SU-Hx rats. While MCT-treated rats do not develop plexiform lesions, the model developed severe PAH and assisted in the validation of PAH therapies. Adult rats are an ideal choice of species for molecular imaging studies for PAH, having sufficiently large lungs to permit good spatial and anatomic resolution in co-registered PET-CT images.

Data from human and experimental PAH tissues confirm enhanced expression of VEGF-A in pulmonary vascular lesions, suggesting that a sensitive probe of VEGF-A expression could be used to monitor PAH disease. The ability of bevacizumab to recognize VEGF-A in human and experimental rat PAH tissues, coupled with its extensive record of use as an approved therapy and investigational use as a human imaging probe, suggests a rapidly translatable strategy for imaging human PAH and an efficient means for validating its utility in two accepted pre-clinical models of PAH.

⁸⁹Zr-bevacizumab is a sensitive PET imaging probe of VEGF-A expression in human and experimental tumor vascularization. ⁸⁹Zr is a radioisotope of zirconium with a half-life of about 78 hours and has enabled molecular imaging applications via conjugation to antibody probes. Bevacizumab has been previously conjugated with ⁸⁹Zr for use as a positron emission tomography (PET) imaging probe of tumor angiogenesis. For example, ⁸⁹Zr-bevacizumab sensitively detects human SKOV3 ovarian tumor xenografts in nude mouse models by binding avidly to stromal-derived VEGF in the tumor vasculature and tracks the regression of tumor xenograft mass and vascularization of cisplatin-resistant ovarian carcinoma in response to molecular targeted therapies to HSP90. ⁸⁹Zr-bevacizumab was shown to track the clinical regression of renal cell carcinoma in human subjects following treatment with anti-angiogenic adjuvant therapies and permit the visualization of VEGF-A expression in human breast carcinoma primary tumors. Thus, ⁸⁹Zr-bevacizumab appears to have favorable characteristics and high sensitivity as a PET-CT probe for detecting enhanced angiogenic activity in the tumor vasculature of experimental and human cancer.

Studies were conducted to demonstrate the feasibility of ⁸⁹Zr-bevacizumab as an imaging probe of angiogenic activity in experimental PAH. Data confirmed that VEGF-A is overexpressed in the vascular lesions of human PAH, as well as remodeled vessels of rats with experimentally induced PAH. In these studies, it was found that bevacizumab sensitively detected the expression of rat VEGF₁₆₅, both as an isolated protein and in the context of vascular lesions of SU-Hx- and MCT-induced PAH in rats. Given the viability of ⁸⁹Zr-bevacizumab as a PET-CT probe of angiogenic activity in animals and in man, ⁸⁹Zr-bevacizumab could similarly detect the angiogenic signaling present in rats with experimental PAH. ⁸⁹Zr-conjugated bevacizumab was generated under a current good manufacturing practices (cGMP) compliant protocol. Briefly, antibody was reacted with a bifunctional tetrafluorophenyl-N-succinyl-desferal-Fe chelating group to yield a conjugate with an average of two substitutions per antibody, demonstrated by HPLC. Chelated Fe⁺³ was then displaced with ⁸⁹Zr⁺⁴ to yield the conjugated probe. A sensitive ELISA demonstrated unaltered VEGF binding in labeled versus unlabeled material, confirming the affinity for VEGF was generally not disrupted by conjugation.

Rats were subjected to SU-Hx for about 3 weeks, followed by normoxia for about 2 weeks, with the presence of PAH confirmed non-invasively by the presence of shortened pulmonary arterial acceleration times by cardiac ultrasound in PAH animals compared to control animals. In a subset of animals, treatment with a dual vasodilator regimen consisting of about 5 mg/kg/d po ambrisentan and about 10 mg/kg/d tadalafil was administered by oral gavage during the normoxic phase of the protocol. Each animal was injected via the tail vein with about 7.4 MBq/0.2 mCi of activity in about 200 μg ⁸⁹Zr-bevacizumab, with a specific activity of about 37 MBq/mg or about 1 mCi/mg in a volume of about 50 μL normal saline, as an approximately 30 MBq/kg or 0.8 mg/kg i.v. single dose. This dose was relatively higher by weight than doses previously used in human tumor imaging applications, which employed an about 37-100 MBq or about 1-3 mCi dose via about 5-8 mg of labeled antibody or approximately 0.6-1.6 MBq/kg or 0.08 mg/kg i.v. single doses. The dose administered to rats was chosen based on the proportionally decreased sensitivity of bevacizumab for rat vs. human VEGF₁₆₅ (see FIG. 2).

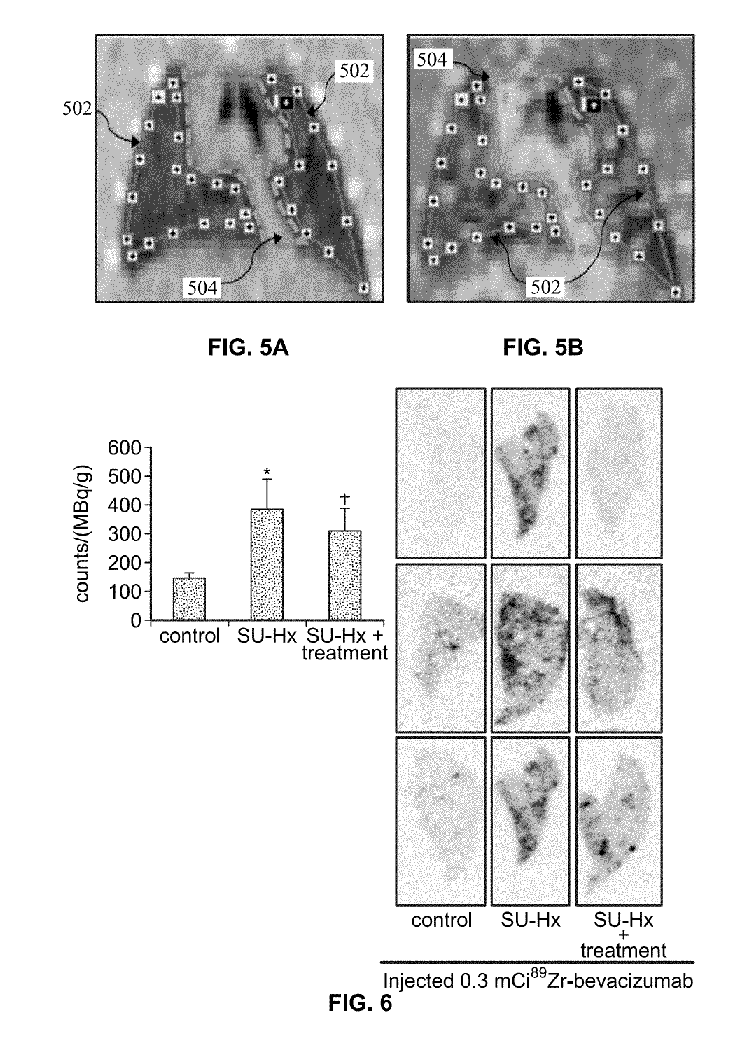

The animals were scanned on days 0, 2, 4, and 7 following injection. Overall lung signal intensity was highest on days 2-4, with activity in control animals localized to great vessels and heart, consistent with significant blood pool activity seen in prior studies (see FIG. 4). FIG. 4 shows representative PET-CT scans demonstrating enhanced ⁸⁹Zr-bevacizumab signals in the peripheral lung fields in rats subjected to SU5416 and hypoxia (SU-Hx, 402) versus control rats 404. Healthy rats injected with ⁸⁹Zr-bevacizumab demonstrated blood pool signal primarily concentrated over the heart and the thoracic aorta 406, whereas rats with experimental PAH showed uptake in the peripheral areas lung fields 408 not seen in the controls. In the SU-Hx treated rats, relatively increased activity was consistently observed in the periphery of the lungs on cross-sectional and coronal co-registered PET-CT images of the chest. This pattern of ⁸⁹Zr-bevacizumab retention was suggestive of an enhanced probe signal in the mid-distal pulmonary vasculature. Regions of interest (ROIs) were defined on computed tomography (CT) to distinguish the peripheral lung from mediastinum (see FIGS. 5A-5B) blinded with respect to PET data. In preliminary studies with limited animal numbers (n=3-4 per group), a trend towards an about 3.1-fold increase in ratio of standard uptake values (SUVs) for peripheral lung versus mediastinal ROIs was observed in SU-Hx rats (about 1.53±1.20) as compared to control rats (about 0.49±0.09, p=0.07).

In FIGS. 5A-5B, representative ROIs used to define peripheral lung fields 502 and mediastinal structures 504 were drawn using CT images (FIG. 5A) blinded with respect to PET data (super-imposed with CT, FIG. 5B). Mean SUVs were calculated for peripheral lung and mediastinal volumes, and SUV ratios (SUVR) of peripheral:mediastinal ROIs were determined. Preliminary analysis of SU-Hx versus control rats revealed a trend towards increased SUVRs in SU-Hx treated rats of about 1.53±1.20 versus a ratio of about 0.49±0.09 in control rats, p=0.07, n=3-4 per group.

To confirm enhanced retention of ⁸⁹Zr-bevacizumab independent of blood pool, animals were euthanized and lungs flushed of blood via saline infusion through the right ventricle at about 100 cm H₂O for about 1 minute, followed by infusion of about 1% PFA at about 100 cm H₂O, and intratracheally at about 20 cm H₂O for about 5 min. Frozen sections (about 10 μm) of the right lower lobe were subjected to autoradiography. Diseased animals were notable for an about 2.6-fold, statistically significant increase in radioisotope detection as compared to control animals (see FIG. 6) and in a pattern that appeared to highlight the peripheral vs. central lung tissues. As shown in FIG. 6, autoradiography of rat lung sections demonstrated enhanced retention of ⁸⁹Zr-bevacizumab among rats with SU-Hx induced PAH, or diseased rats receiving dual vasodilator treatment as compared to control rats, with retention assessed by integrated density adjusted by injected dose and weight (counts/Mbq/g). *p=0.01 vs. control, †p=0.02 vs. control, NS vs. SU-Hx; n=3-4 per group.

In these studies, despite administration of a potent dual vasodilator regimen (ambrisentan about 5 mg/kg/d and tadalafil about 10 mg/kg/d) in the treatment group, there was no significant difference in the retention of ⁸⁹Zr-bevacizumab in treated versus untreated animals with PAH. Taken together, these results suggest that (1) ⁸⁹Zr-bevacizumab uptake reflects enhanced VEGF expression in the distal vascular beds of diseased animals, and (2) treatment with two potent vasodilators does not significantly modify the process detected by the ⁸⁹Zr-bevacizumab probe.

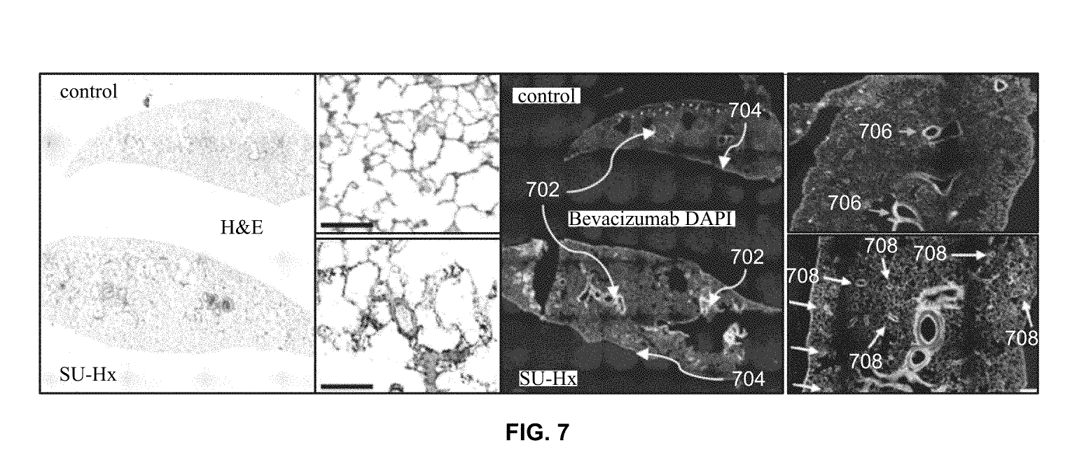

To confirm the former concept, lung tissues were examined by immunohistochemistry to ascertain the retention and distribution of bevacizumab after in vivo administration (see FIG. 7). Using fluorescent anti-human IgG, retained bevacizmab was detected only in central lobar arteries and lobar bronchi of control lungs. Lungs from SU-Hx-treated rats demonstrated retention of bevacizumab throughout central and peripheral lung tissues and appeared to stain a large portion of small, distal arterioles of less than about 50 μm in diameter, consistent with the staining of bevacizmab and a pan-species anti-VEGF-A mAb in lung sections from SU-Hx and MCT-treated rats (see FIGS. 1 and 3). These data demonstrate that ⁸⁹Zr-bevacizumab uptake reflects enhanced VEGF-A expression in the distal vasculature of diseased animals, visualized by 3D PET scanning in vivo, and confirmed by autoradiography and immuno-fluorescence of lung sections ex vivo.

As shown in FIG. 7, histology reveals neointimal and complex lesions of medium (less than about 50 μm) vessels in SU-Hx but not control rat lungs (left panels, inset bars=100 μm). Immunohistochemistry performed on rats injected in vivo with ⁸⁹Zr-bevacizumab (about 0.3 mCi, about 200 μg) detected retained bevacizumab (anti-human IgG 702, DAPI 704) in the central large vessels and airways of control rat lungs 706 (bars=100 μm), as well as SU-Hx treated rats, but demonstrated enhanced IgG retention 708 in SU-Hx rats in small and medium sized vessels throughout the peripheral lung tissues.

In some embodiments, the probe in the experimental PAH models is optimized in various ways. For example, in one embodiment, doses of probe range from about 2-50 MBq/kg in SU-Hx rats and controls to optimize sensitivity and specificity for disease, based on differences in the SUV ratios of peripheral:mediastinal structures. Similarly, in other embodiments, scans are performed at day 2, 4, and 7 following administration to optimize an ideal washout period. Extrinsic and intrinsic respiratory gating protocols are compared to ungated studies to determine whether or not anatomic resolution and specificity may be gained. The impact of variable PET acquisition times on sensitivity and anatomic resolution are tested systematically, while CT scanning times are generally minimized to limit additional exposure.

In one embodiment, the relationship of disease progression, severity, and survival to VEGF imaging in PAH are ascertained by examining serial changes in ⁸⁹Zr-bevacizumab PET imaging in response to clinical parameters such as exercise function (e.g., 6 minute walk test) and invasive hemodynamic measurements. PET imaging may reflect changes in clinical disease activity that will precede or predict changes in traditional clinical assessment parameters. Thus, PET imaging using this reagent may allow more rapid and efficient titration of medication or identification of efficacious agents or indicate that patients with early symptoms and equivocal traditional testing should be started earlier on medical therapy; alternately, PET imaging results might identify particularly high-risk individuals or individuals refractory to therapy, who might benefit from more aggressive interventions such as lung transplantation. The assertions with respect to ⁸⁹Zr-bevacizumab PET imaging are: (1) pulmonary vascular VEGF activity reflects the development, progression, and severity of experimental PAH; and (2) pulmonary vascular VEGF activity may increase before the development of hemodynamically significant PAH.

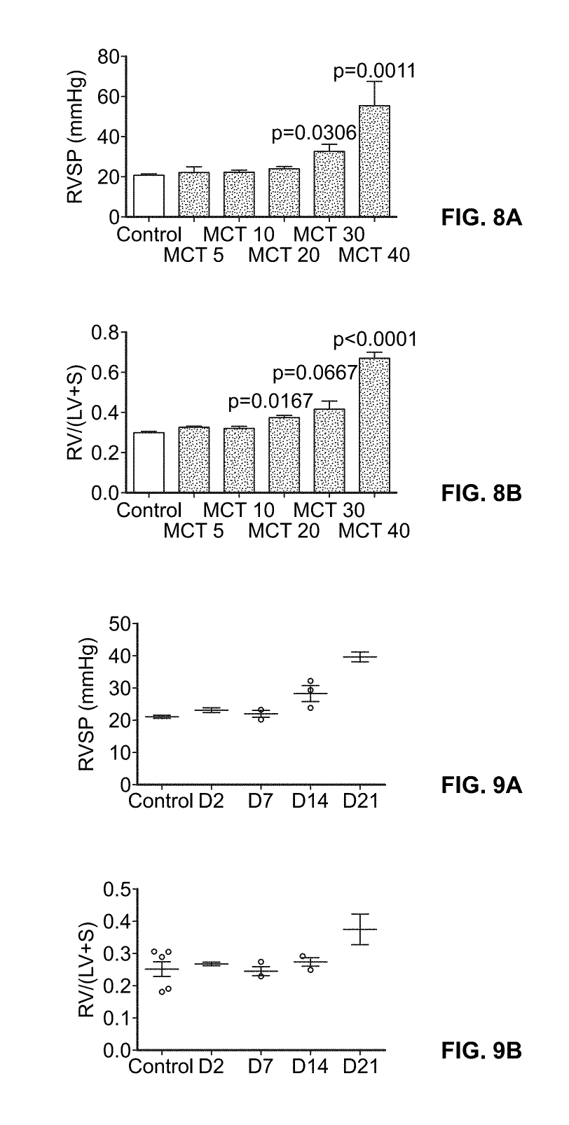

The severity of disease in each model may be modulated according to the following process. Adult rats are administered varying single doses of MCT at about 20, about 30, and about 40 mg/kg s.c. to elicit varying degrees of PAH and right ventricular hypertrophy (see FIGS. 8A-8B) and monitored for PAH and other physiologic changes by telemetry over time. As shown in FIGS. 8A-8B, variable single doses (about 5-40 mg/kg s.c.) of monocrotaline (MCT) administered to adult Sprague-Dawley rats result in varying degrees of PAH at about 3 weeks based on right ventricular systolic pressure measurements (RVSP, FIG. 8A), as well as variable degrees of right ventricular hypertrophy, expressed as Fulton's ratio (RV/(LV+S), FIG. 8B, p values versus control shown, n=4 per group).

In the SU-Hx model, adult rats are administered a standard dose of SU5416 (about 20 mg/kg s.c.), and/or about 3 weeks of normobaric hypoxia (FIO₂=0.10), alone or in combination, followed by about 6 weeks of normoxia, to yield three groups with varying degrees of PAH, as previously described. Serial ⁸⁹Zr-bevacizumab PET-CT scans are performed at day 0, 7, 14, 21, 28, 35, and/or 42 following initial treatment with MCT or upon completion of exposure to SU5416 and/or hypoxia, based on previous characterization of the kinetics of PAH development in these models (MCT shown in FIGS. 9A-9B). FIGS. 9A-9B illustrate the development of elevated right ventricular systolic pressure (RVSP) and right ventricular hypertrophy (RV/(LV+S)) as a function of time following a single dose of about 40 mg/kg s.c. monocrotaline. PAH first manifests after day 14, whereas right ventricular hypertrophy is evident at day 21. PET imaging of ⁸⁹Zr-bevacizumab is anticipated to reveal early changes in the expression of VEGF-A in the distal circulation of humans with PAH and animal models before the onset of frank elevation in RVSP and RVH.

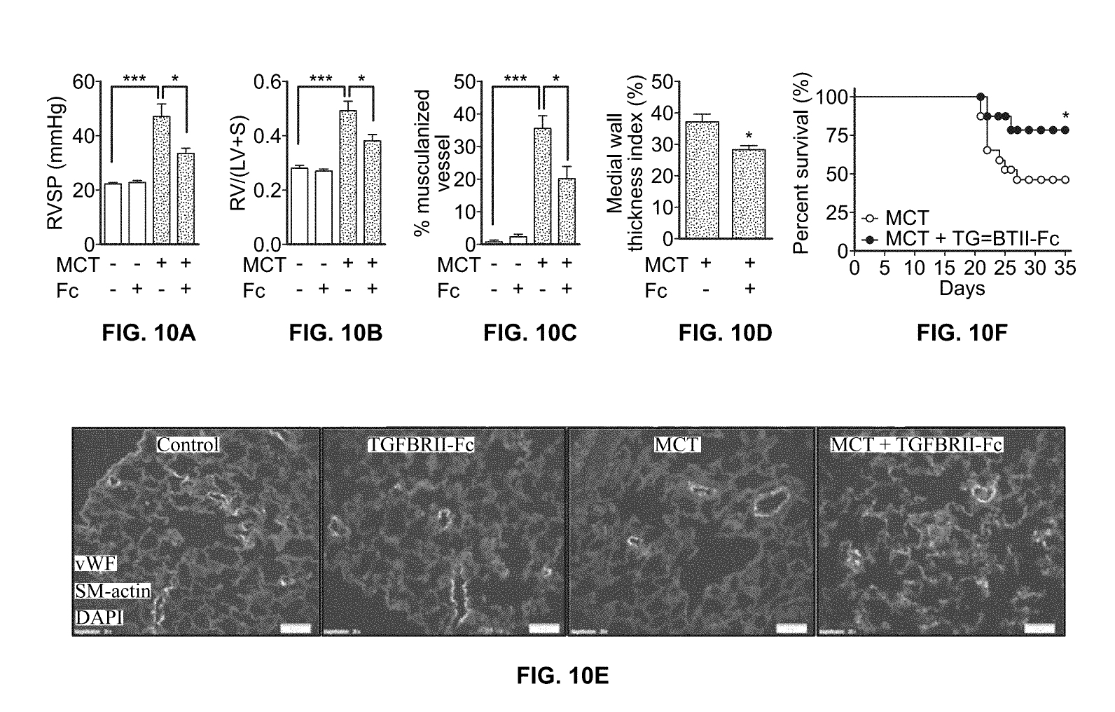

In some embodiments, ⁸⁹Zr-bevacizumab PET imaging may be used to reveal the impact of disease-modifying therapies. As shown in FIGS. 10A-10E, adult rats treated with MCT (about 40 mg/kg s.c.) were administered TGFBRII-Fc (about 15 mg/kg IP twice weekly) or vehicle for about 3 weeks. TGFBRII-Fc significantly attenuated RVSP (see FIG. 10A) and RV hypertrophy (see FIG. 10B) in comparison to vehicle (n=6-8) and decreased the percentage of fully muscularized vessels (about 10-50 μm diameter) and medial wall thickness (see FIGS, 10C, 10D). TGFBRII-Fc treatment reduced muscularization, evident by smooth muscle actin staining of vWF⁺ small vessels (see FIG. 10E, bar=50 μm). In additional studies, delayed treatment with TGFBRII-Fc starting at about 2.5 weeks after MCT improved survival (see FIG. 10F). Data expressed as mean ±SEM, *p<0.05 and ***p<0.001 as indicated. As an example of a potentially disease-modifying therapy, the impact of currently experimental interventions such as the TGF-β ligand trap TGFBRII-Fc might be demonstrated in changes in the pattern of ⁸⁹Zr-bevacizumab PET imaging, whereas conventional vasodilator therapies such as ambrisentan and tadalfil do not appear to have an effect, as shown, e.g., in FIG. 6.

The gathered data predicts that the hemodynamic severity of PAH measured by serial invasive hemodynamic assessments or implantable telemetry devices measuring right ventricular or pulmonary artery pressures (progressing over time in humans, according to natural history of disease, or altering in response to therapy, or varying in response to different degrees of exposure to environmental or other insults, or in animal models, varying in response to treatments with MCT or SU5416 +/− hypoxia) will correlate closely with SUV ratios of ⁸⁹Zr-bevacizumab in peripheral lung tissues versus blood pool. The absolute SUV in the peripheral lung tissue ROIs are expected to generally increase in proportion to hemodynamic severity. The ability of ⁸⁹Zr-bevacizumab PET to discriminate between treatment and control populations or groups of varying hemodynamic severity could be evaluated by one-way analysis of variance (ANOVA) comparing respective SUV ratios, or, alternately, by defining positive and negative studies in relation to median SUV ratios and performing receiver operating characteristic analysis (ROC) for a given treatment group or hemodynamic severity versus controls. In analyzing the kinetics of development of PAH by serial invasive hemodynamic testing or telemetry, VEGF imaging intensity will generally increase on serial scans before the development of hemodynamically significant PAH. If peripheral lung VEGF-imaging SUV ratios increase prior to frank PAH, VEGF signals in this pre-hypertensive state may predict the severity of subsequent PAH. Varying degrees of ⁸⁹Zr-bevacizumab PET imaging intensity may result due to different stages of disease, different burdens of disease in man, or differences in exposure to PAH-inducing stimuli such as anorexigens or differences in severity of associated disease states, all of which are sources of phenotypic variability that may be reflected by ⁸⁹Zr-bevacizumab PET imaging signal intensities or anatomic distribution.

Based on extensive preliminary data demonstrating the sensitivity of bevacizumab for detecting rat VEGF₁₆₅ in vitro, in vivo, and ex vivo, bevacizumab was found to have sufficient selectivity and affinity for rat VEGF₁₆₅ to permit the detection of VEGF expression in rat pulmonary vasculature. In one embodiment, the specificity of these findings are ensured further by using ⁸⁹Zr-labeled pooled human IgG as a control probe, generated using an identifical procedure to ⁸⁹Zr-bevacizumab. The sensitivity of bevacizumab for rat VEGF is within one order of magnitude of its sensitivity for human VEGF but has approximately 50-fold less sensitivity for murine VEGF₁₆₄, making murine PAH models less ideal. Despite the relatively lower affinity for murine VEGF, bevacizumab was observed to partially block the activity of endogenous VEGF in several murine models. As additional assurance of the specificity of these findings, in some embodiments, alternate rodent- and human-cross reactive anti-VEGF monoclonal antibody, B20-4.1.1 (obtained from Genentech) are used for a set of in vivo and ex vivo experiments. This antibody binds human and rodent VEGF with comparable affinity to bevacizumab for human VEGF₁₆₅ and similarly exerts potent anti-angiogenic effects in multiple rodent models. This antibody is predicted to confirm the findings obtained using ⁸⁹Zr-bevacizumab in rats and help assure the translatability of the findings.

In one embodiment, to further ascertain patterns of VEGF expression in human PAH, the anatomic localization and expression of VEGF is analyzed in a diverse set of lung tissues with and without PAH. VEGF expression in lungs tissues with diverse etiologies of Group I PAH undergoing transplantation, patients with parenchymal lung disease without PAH, and control lung tissues obtained from unaffected adjacent tissues during lung resection for cancer are examined.

Given the heterogeneity of human PAH disease, even within WHO Group I, the degree of VEGF expression in the lung vasculature may vary considerably. The severity of PAH may be a key factor and may correlate staining intensity with pre-transplant hemodynamics and functional status in tissues from PAH patients, as these attributes vary despite tissues being obtained from end-stage disease. Etiology-specific differences in VEGF-A expression, i.e., in HPAH/IPAH versus scleroderma associated PAH, may be found. Enhanced expression of VEGF in vascular lesions and remodeled small vessels are generally consistent findings in Group I PAH but not in controls or severe COPD without PAH.

Emphysema has been variably reported to exhibit decreased VEGF in alveolar septal endothelial cells and brochiolar epithelium or enhanced VEGF expression in bronchiolar smooth muscle and epithelium. These tissues may, therefore, be an important test of the anatomic specificity of VEGF overexpression in PAH. Based on, e.g., the results shown in FIG. 1, enhanced luminal expression of VEGF may be associated with neointimal or plexiform lesions, whereas completely obstructed vessels appear to lack VEGF expression—findings that suggest that VEGF signifies an active versus a completed remodeling process. Interestingly, animal models exhibited distinct VEGF localization, with primarily medial staining in MCT rats, versus intimal, medial, and adventitial staining in SU-Hx-treated rats (see FIGS. 1, 3). Similarly distinct sub-phenotypes for VEGF expression may exist among Group I HPAH, IPAH, and scleroderma patients. Peri-vascular changes may be found in PAH associated with connective tissue disease versus IPAH and HPAH.

In some embodiments, differences in the intensity or anatomic distribution of VEGF among distinct etiologies of PAH may be found and subtle differences or overlap in VEGF expression between PAH and other respiratory and airway diseases such as emphysema or asthma may be discerned by the intensity or anatomic distribution of PET imaging signal.

.

As described above, bevacizumab has a high affinity for human VEGF-A and a very distinct pattern of binding in human PAH tissues (see, e.g., FIG. 1). It is contemplated that enhanced ⁸⁹Zr-bevacizumab retention may be observed in remodeled small and medium sized vessels and vascular lesions of PAH lungs, whereas control lungs generally exhibit retention primarily in large vessels. Enhanced retention of ⁸⁹Zr-bevacizumab in the alveolar septal endothelium of lungs with COPD/emphysema without PAH may occur, but at a generally lower level of intensity and with generally more variably than seen in PAH, allowing one to distinguish between these pathologies radiologically. Validation of VEGF Imaging as a Monitor of Disease-Modifying Therapy in PAH.

The potential impact of disease-modifying therapy on molecular VEGF imaging using serial ⁸⁹Zr-bevacizumab PET imaging and continuous invasive hemodynamic monitoring in rat models of PAH was tested. It was surmised that VEGF imaging would be closely coupled to disease regression with the use of potent and novel therapies that reverse remodeling by augmenting endothelial BMP signaling or by trapping TGF-β but would be less responsive to conventional therapies that act primarily as vasodilators.

This data demonstrates the utility of a novel PET probe for the diagnosis and management of human PAH. It is contemplated that probe activity is closely linked to disease progression or regression. It was surmised that ⁸⁹Zr-bevacizumab PET imaging could help identify pre-morbid disease, guide tailored therapy, and/or provide a criterion for evaluating novel treatments with the potential to modify the natural history of disease. This modality may be useful in defining new sub-phenotypes of PH disease at presentation or during the course of treatment and, thus, in enabling novel paradigms for rational therapy in PAH.

TGFBRII-Fc and BMP9 are novel treatments that address dysregulated TGF-β and BMP signaling in PAH. Heritable PAH in humans is associated with loss-of-function mutations in the bone morphogenetic protein (BMP) type II receptor, and deficient vascular BMP signaling is also observed in other etiologies of Group I PAH and experimental PAH. In human and animal models, this deficiency of BMP receptor-mediated signaling is accompanied by overexpression of TGF-β ligands and excessive TGF-β signaling. _ENREF_38 The imbalance of BMP versus TGF-β signaling in PAH is a theme that has prompted a number of therapeutic strategies for addressing these signaling defects. For example, systemic BMP9 therapy is used as a strategy for ameliorating pulmonary vascular remodeling and experimental PAH, rescuing the loss-of-function in endothelial BMP signaling and BMP type II receptor expression in SU-Hx rats, MCT-treated rats, and SU-Hx mice. This therapy induces regression of PAH even in established disease and with favorable tolerability. A soluble TGF-β ligand trap utilizing the TGF-β type II receptor expressed as an Fc fusion protein (TGFBRII-Fc) similarly improves pulmonary vascular remodeling and PAH in MCT-treated rats (see FIGS. 10A-10E). Administration of TGFBRII-Fc improves vascular remodeling even with established disease, and, in this context, significantly improves survival of animals challenged with an ˜LD₅₀ dose of MCT (see FIG. 10F). Importantly, neither of these interventions acts by modulating vascular tone but, rather, attempt to address underlying signaling abnormalities to modulate the process of vascular remodeling, either by attenuating endothelial apoptosis (BMP9) or by attenuating myogenic TGF-β signaling in the vascular wall (TGFBRII-Fc). The embodiments described herein examine these strategies as disease modifying agents, showing that PET-visualized vascular overexpression of VEGF-A via ⁸⁹Zr-bevacizumab will improve following the use of interventions that directly affect vascular remodeling and improve in advance of hemodynamic changes that are likely to occur in a delayed fashion.

It is contemplated that classes of medication that have the potential to modify pulmonary vascular remodeling and angiogenic activity also affect VEGF expression in the vasculature. In addition to being potent vasodilators, prostacyclin and ETRA may exert anti-mitogenic, anti-fibrotic, and/or anti-inflammatory effects in the vasculature and thereby assist in modifying remodeling and/or angiogenic activity in PAH models. Despite this theoretical mechanism of action, studies failed to show a significant impact of combined ETRA and PDE5 inhibition therapies on ⁸⁹Zr-bevacizumab retention by autoradiography (see, e.g., FIG. 6). Potent anti-remodeling agents such as recombinant BMP9 or TGFBRII-Fc may exert more significant effects on remodeling and angiogenic signaling and, therefore, impact VEGF-A imaging more than conventional vasodilator medications.

Abnormal VEGF-A expression generally occurs in the context of other airway and vascular lung diseases. VEGF-A expression is altered in a tissue and cell-specific manner in emphysema, with variable reports of decreased or increased expression in bronchial smooth muscle, bronchial epithelium, and alveolar endothelial cells in COPD and associated cigarette use. VEGF contributes mechanistically to airway integrity, as disruption of VEGF signaling results in emphysema. In the context of its application for PAH, it is contemplated that COPD and emphysema represent potential confounders for the interpretation of ⁸⁹Zr-bevacizumab PET imaging. However, studies in human tissues,and in MCT versus SU5416-treated rats reveal potential unique imaging and histological VEGF phenotypes associated with emphysematous disease, which may have similar utility for monitoring COPD activity and therapy. Alternatively, small arteriolar involvement may be a finding that is specific to PAH, and its presence may reflect whether or not emphysema is accompanied by secondary or WHO Group 3 PAH.

Bevacizumab has extensive tolerability and safety data. Bevacizumab is administered chronically as an adjuvant in the treatment of solid tumors with good tolerability. In retrospective analyses of several thousand patients, the most common bevacizumab-related toxicities included hypertension (about 5.3-22.0%), bleeding (about 2.2-3.0% of patients), arterial thromboembolism (about 1.0-2.3%), proteinuria (about 1.0%), and wound healing complications (about 1.0%). Bevacizumab is typically administered until primary or secondary relapse with doses of about 5-10 mg/kg i.v. about every 2 weeks. Toxicity is generally related to premorbid conditions and surgical trauma and is generally dependent upon dose and duration. Importantly, bevacizumab has been used investigationally at lower systemic doses (less than about 2 mg/kg i.v.) or via local administration for non-oncologic disease, including intravitreal administration for age-related macular degeneration and low-dose systemic or intranasal therapy for arteriovenous malformations in hereditary hemorrhagic telangiectasia (HHT), in both applications with excellent tolerability and efficacy.

In one embodiment, a single dose of about 0.08 mg/kg bevacizumab is used for PET imaging of lungs in PAH, a quantity that is about 1% of a typical therapeutic dose. This dose of bevacizumab for human imaging represents about 1/3,000^{th} of the aggregate exposure of cancer adjuvant therapy over about 2 years. The very small exposure to bevacizumab for imaging generally very well-tolerated and has not been associated with toxicity in other human imaging applications.

The proposed radioisotype dose of ⁸⁹Zr-bevacizumab for human applications is about 37 MBq/1 mCi, corresponding to an exposure of approximately 25 mSv, which is similar to the exposure received in a dual isotope cardiac perfusion stress test, or a single vessel percutaneous coronary intervention. Radiation toxicity in animals from single or repeated injections of ⁸⁹Zr-bevacizumab has not been observed or previously reported.

In some embodiments, ⁸⁹Zr-bevacizumab serves as a non-invasive measure of pulmonary vascular remodeling activity in experimental PAH. The following factors are considered important in optimizing the use of ⁸⁹Zr-bevacizumab: (1) optimizing dosing, administration, and data acquisition for ⁸⁹Zr-bevacizumab PET imaging; (2) demonstrating sensitivity and specificity of the ⁸⁹Zr-bevacizumab peripheral:mediastinal SUV ratio; (3) demonstrating kinetics and coupling to disease severity of ⁸⁹Zr-bevacizumab PET imaging; (4) demonstrating specificity of ⁸⁹Zr-bevacizumab uptake in ex vivo perfused PAH lung tissues; and/or (5) demonstrating the sensitivity of ⁸⁹Zr-bevacizumab PET imaging for disease regression.

In summary, PH continues to carry a dire prognosis despite current therapies, which is further challenged by recent data suggesting that PH is far more prevalent and underdiagnosed than previously appreciated. The data described herein strongly support feasibility and utility of using VEGF-A as a target of molecular imaging to monitor angiogenic activity in the pulmonary arterioles by PET-CT imaging. Dysregulated angiogenic signaling and VEGF-A overexpression are believed to be closely linked to PAH disease. Demonstrating the utility of ⁸⁹Zr-bevacizumab, a rapidly translatable imaging strategy for PAH, will have very high impact by addressing several of the most important barriers to improving disease outcomes, by providing a modality for earlier diagnosis, by providing direct monitoring of disease activity, and by assisting with evaluation of novel, potentially disease-modifying agents. The success of this strategy could spur the development of other molecular imaging modalities directed towards other targets which contribute to this disease.

Methods:

cGMP production of ⁸⁹Zr-bevacizumab. Bevacizumab (about 25 mg/mL, Genentech, San Francisco, Calif.) was purified from other excipients with centrifugal concentrators (Vivaspin-2, Sartorius, Göttingen, Germany), diluted in sterile water at about 10 mg/mL. Bevacizumab was reacted with the bifunctional chelate TFP-N-sucDf-Fe (ABX GMbH, Radeberg, Germany) at about room temperature for about 30 minutes at a pH of about 9.5-10.0 (about 0.1M Na₂CO₃) at a molar ratio of about 2 chelating groups per Ab molecule. After conjugation, the mixture was set to a pH of about 4.0-4.4 (about 0.25 mol/L H₂SO₄), and 460 μl of 25 mg/ml EDTA was added. The solutions were mixed at room temperature for about 30 minutes and purified by centrifugal ultrafiltration 5 times in sterile water. The resulting material (N-sucDf-bevacizumab) was diluted to about 10 mg/ml, verified by HPLC. Radiolabeling was performed with [⁸⁹Zr]-oxalate (IBA Molecular, Richmond, Va.) adjusted to a pH of about 6.5-7.0 with about 200 μL 1M oxalic acid, about 400 μl of about 0.9% NaCl, about 90 μl of about 2M Na₂CO₃, and about 1 ml of about 0.5M HEPES. About 250 μl of N-sucDf-BEV was added to the resulting solution and mixed for about 60 minutes at about 550 rpm. The product was purified again by centrifugal ultrafiltration into about 0.9% NaCl. A sensitive ELISA (Q-BEVA, Matriks Biotek, Turkey) was used to confirm the ability of bevacizumab to bind VEGF following conjugation to N-SucDF and following radiolabeling. This kit was designed to quantitate biologically active bevacizumab in serum and plasma samples. Unmodified bevacizumab, N-sucDf-bevacizumab, and ⁸⁹Zr-bevacizumab were incubated with microtiter wells adsorbed with human VEGF. After washing, biotin conjugated hVEGF was added to detect free valencies of captured bevacizumab and then washed and developed with streptavidin-HRP, confirming the VEGF binding capacities of bevacizumab, N-SucDF-bevacizumab, and ⁸⁹Zr-bevacizumab were generally equivalent.

In vivo imaging of rats and quantitation of VEGF signaling intensity. In one example of in vivo studies of diseased and control rats, PET-CT imaging is performed with a GE eXplore VISTA scanner with an imaging field of view of about 6 cm. Two crystals, LYSO and GSO with distinct scintillation decay times are used, with 1.5 mm wide crystals used to generate high resoluation images, about 1.6 mm from the center of the field of view and with yields of approximately 4% count sensitivity. ⁸⁹Zr-bevacizumab signal intensities are calculated for two principal regions of interest (ROI) by the standard uptake value (SUV) method, where mean image-derived radioactivity C(t) over the ROI at time t is divided by the ratio of the injected activity extrapolated to time t to animal body weight. An SUV ratio (SUVR) is calculated based on the SUV_{peripheral lung} for an ROI defined as the intrathoracic space excluding heart and mediastinum to represent the peripheral lung tissue, divided by the SUV_{blood pool} for an ROI defined by the mediastinum and heart to represent the blood pool (see, e.g., FIG. 5). These ROI are selected on coronal and cross-section planes of a given study by a blinded investigator using CT images without access to PET imaging data. For experiments analyzing VEGF signal intensities following ⁸⁹Zr-bevacizumab perfusion of explanted human lungs, a similar SUVR is calculated based on the SUV for an ROI corresponding to peripheral right upper lobe tissues divided by the SUV for an ROI corresponding to the lobar and subsequent second and third subsegmental arteries and veins, again guided by a blinded investigator using CT images without PET imaging data.

In some embodiments, motion artifacts due to cardiac and respiratory cycles are compensated by intrinsic or triggered gating. In animal models, for example, respiratory gating is triggered by ventilating rats intratracheally (about 16 ga. angiocath), with volume control ventilation with a tidal volume of about 12 mL/kg at a frequency of about 10/min, while maintaining anesthesia with about 1% inhaled isoflurane, using this cycle to identify images obtained at end tidal volume during reconstruction. Alternatively, CT derived raw imaging data are used to generate an intrinsic gating signal to rearrange projection images during reconstruction into image sets specific to stages of the respiratory cycle under mechanical ventilation. In addition to respiratory gating, the cardiac cycle is monitored by surface EKG, which is used to generate reconstructed images from end diastolic images obtained at the initial deflection of each QRS complex. Since scan times are limited, tradeoffs in scanning efficiency versus gating need to be determined, weighing effects of total acquisition times on sensitivity versus improvements in spatial and anatomic resolution afforded by gating.

In one process, lung sections are fixed in about 1% PFA in PBS overnight and transferred to about 30% sucrose-PBS and embedded in OCT, with samples from diseased and healthy animals, and animals not receiving ⁸⁹Zr-bevacizumab embedded side by side as internal controls to ensure comparable thickness upon sectioning. Frozen sections are cut at about 10 μm and captured on Lysine coated slides. To quantitate activity by autoradiography, slides are placed on a phosphor plate (Kodak SO230) and exposed for about 14 hours. An about 50 μm resolution digital image is obtained using a phosphor reader (Personal Molecular Imager, Bio-Rad, California). For immunohistochemistry and immunofluorescence, frozen sections are post-fixed with about 1% PFA in PBS for about 5 minutes and then washed and blocked according to recommended protocols. For bevacizumab staining of rat tissues, about 10-25 μg/mL of primary antibody may be used, followed by Alexa Fluor 488 conjugated F(ab′)2-goat anti-human IgG (Life Technologies, A11017, 1:100). For pan-species VEGF staining, about 10 μg/mL of rabbit mAb anti-VEGF-A (Abcam, ab46154, 5-10 μg/mL) may be used followed by Alex Fluor 488 conjugated F(ab′)2 goat anti-rabbit IgG (Life technologies, A11070, 1:100).

Several uses of ⁸⁹Zr-Bevacizumab for the detection of disease activity in pulmonary arterial hypertension are contemplated. For example, ⁸⁹Zr-Bevacizumab may be used to detect early disease-related remodeling activity in the pulmonary arteriolar circulation prior to (1) the appearance of other clinical signs or symptoms, or the development of echocardiographic, magnetic resonance imaging (MRI), CT, and/or invasive hemodynamic measurement abnormalities showing elevated filling pressures in the pulmonary arterial circuit, or (2) evidence of end organ changes such as dilatation of the proximal pulmonary artery or dilatation, reduced ejection fraction, or hypertrophy of the right ventricle.

⁸⁹Zr-Bevacizumab may also be used to monitor active remodeling of the pulmonary arteriolar circulation, and monitor the efficacy of specific interventions. Disease activity may be modulated by correcting or improving the underlying contributing factors in conditions in which this may be possible, such as by providing therapy for the underlying connective tissue disease, liver failure, intracardiac or left-to-right shunt, infection (such as HIV), or the like. Alternatively, disease activity may be modulated by current or novel treatments that treat underlying mechanisms of disease, including currently accepted medications for pulmonary arterial hypertension such as prostacyclin and prostacyclin analogs, phosphodiesterase 5 inhibitors, calcium channel blockers, endothelin receptor antagonists, and/or soluble guanylate cyclase activating agents. Novel drugs may target inflammatory processes, metabolic changes, cell proliferation, and growth factor, cytokine, or chemokine signaling in order to modify disease. The potential success or clinical response of these agents may be monitored in a much more rapid fashion using a molecular probe for pulmonary arteriolar remodeling activity, whereas conventional endpoints such as 6 minute walk distance, invasive hemodynamics, echocardiography, and clinical symptoms may take several months to reflect a clinical response.

Alternate radiotracer labels as compared to ⁸⁹Zr, such as ⁶⁸Ga, ¹⁸F, ⁶⁴Cu, ⁸⁶Y, ⁷⁶Br, ¹²⁴I or the like, may be used to label the antibody and may, in some instances, provide favorable sensitivity, signal-to-noise ratio, longevity due to half-life (decreased or increased), and/or improved safety due to decreased radiation exposure to patients and bystanders. The alternative radiotracer labels may be used instead of ⁸⁹Zr based on these characteristics or based on the clinical situation. In addition, some of these labeling agents may be visualized by non-ionizing imaging methods to provide greater correlative anatomic information than possible with PET imaging alone.

Alternate contrast moieties may be used to label the antibody, including but not limited to Gadolinium, Iodine, Iron, or nano-particles containing some of these molecules, to provide the ability to visualize probes via non-ionizing modalities such as CT, MRI, or other imaging modalities. These modalities and contrast moieties could be used instead of, or in addition to, the PET methods proposed. These methods are advantageous in reducing the exposure to ionizing radiation, if used instead of PET imaging methods. If used as a bi-functional or simultaneously administered probe agent with PET visualized agents, these alternate probes provide more detailed, higher resolution anatomic localization to help inform imaging data obtained by PET.

In some embodiments, alternate antibodies than bevacizumab are used to visualize enhanced VEGF expression in the vasculature and lung tissues in pulmonary hypertension. Antibodies that are selective for various VEGF isoforms such as VEGF-a/VEGF₁₆₅ (as in the case of bevacizumab) or other alternatively spliced isoforms (such as human VEGF₁₂₁, VEGF₁₂₁-b, VEGF₁₄₅, VEGF₁₆₅, VEGF₁₆₅-b, VEGF₁₈₉, or VEGF₂₀₆ isoforms) are advantageous for visualizing changes due to pulmonary vascular remodeling, based on the abundance of particular isoforms expressed in the vascular wall.

Alternate linker chemistries may be used to label a given anti-VEGF antibody with radiometals, heavy metals, or other PET, CT, or MRI imaging modalities.

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

1. A method of detecting a disease associated with pulmonary vascular remodeling, the method comprising:
administering a radioisotope-conjugated antibody against vascular endothelial growth factor (VEGF); and
imaging said antibody using positron emission tomography (PET), computed tomography (CT), or magnetic resonance imaging (MIR),
wherein retention of said antibody reflects vascular remodeling.

2. The method of claim 1, wherein the disease is pulmonary arterial hypertension (PAH).

3. The method of claim 1, wherein the radioisotope-conjugated antibody is a humanized monoclonal antibody.

4. The method of any of claim 1, wherein the radioisotope is ⁸⁹Zr, ⁶⁸Ga, ¹⁸F, ⁶⁴Cu, ⁸⁶Y, ⁷⁶Br, or ¹²⁴I.

5. The method of claim 4, wherein the radioisotope-conjugated antibody is ⁸⁹Zr-bevacizumab.

6. The method of claim 1, wherein the administering is to a patient having no detectable echocardiographic, MRI, CT, or invasive hemodynamic measurement abnormalities.

7. A method of monitoring the efficacy of therapeutics or prophylactics for a disease that is associated with pulmonary vascular remodeling, the method comprising:
administering a radioisotope-conjugated antibody against vascular endothelial growth factor (VEGF); and
imaging said antibody using positron emission tomography (PET), computed tomography (CT), or magnetic resonance imaging (MIR),
wherein said imaging reflects the ability of said therapeutics or prophylactics to decrease said vascular remodeling.

8. The method of claim 7, wherein the disease is pulmonary arterial hypertension (PAH).

9. The method of claim 7, wherein the radioisotope-conjugated antibody is a humanized monoclonal antibody.

10. The method of claim 7, wherein the radioisotope is ⁸⁹Zr, ⁶⁸Ga, ¹⁸F, ⁶⁴Cu, ⁸⁶Y, ⁷⁶Br, or ¹²⁴I.

11. The method of claim 10, wherein the radioisotope-conjugated antibody is ⁸⁹Zr-bevacizumab.

12. The method of claim 7, wherein the administering is to a patient having no detectable echocardiographic, MRI, CT, or invasive hemodynamic measurement abnormalities.

13. A method of identifying novel therapeutics for a disease that is associated with pulmonary vascular remodeling, the method comprising:
administering a radioisotope-conjugated antibody against vascular endothelial growth factor (VEGF) and imaging said antibody using positron emission tomography (PET), computed tomography (CT), or magnetic resonance imaging (MIR),
wherein said imaging reflects the ability of said novel therapeutics to decrease said vascular remodeling.

14. The method of claim 13, wherein the disease is pulmonary arterial hypertension (PAH).

15. The method of claim 13, wherein the radioisotope-conjugated antibody is a humanized monoclonal antibody.

16. The method of claim 13, wherein the radioisotope is ⁸⁹Zr, ⁶⁸Ga, ¹⁸F, ⁶⁴Cu, ⁸⁶Y, ⁷⁶Br, or ¹²⁴I.

17. The method of claim 16, wherein the radioisotope-conjugated antibody is ⁸⁹Zr-bevacizumab.

18. The method of claim 13, wherein the administering is to a patient having no detectable echocardiographic, MRI, CT, or invasive hemodynamic measurement abnormalities.

19. A pulmonary vascular biomarker comprising a radioisotope-conjugated antibody, wherein the biomarker is used for detecting a disease associated with pulmonary vascular remodeling or for monitoring the efficacy of therapeutics or prophylactics for a disease that is associated with pulmonary vascular remodeling.

20. The biomarker claim 19, wherein the disease is pulmonary arterial hypertension (PAH).

21. The biomarker of claim 19, wherein the radioisotope-conjugated antibody is a humanized monoclonal antibody.

22. The method of claim 19, wherein the radioisotope is ⁸⁹Zr, ⁸⁶Ga, ¹⁸F, ⁶⁴Cu, ⁸⁶Y, ⁷⁶Br, or ¹²⁴I.

23. The biomarker of claim 22, wherein the radioisotope-conjugated antibody is ⁸⁹Zr-bevacizumab.

24. The biomarker of claim 19, wherein the radioisotope-conjugated humanized monoclonal antibody is directed against vascular endothelial growth factor (VEGF) as a positron emission tomography (PET) imaging probe.

25. The biomarker of claim 19, wherein the disease is pulmonary arterial hypertension (PAH).

26. The biomarker of claim 19, wherein the biomarker is configured to provide expedited screening for pulmonary vascular disease in individuals with normal pulmonary function tests, cardiac stress testing, resting echocardiograms, and resting invasive hemodynamic measurements that are non-diagnostic for PAH.
